(19) United States
(12) Patent Application Publication
(10) Pub. No.: US 2008/0222745 A1
(43) Pub. Date: Sep. 11, 2008

(54) Colcemid-Treatment of Oocytes to enhance Nuclear Transfer Cloning

(75) Inventor: Kenneth L. White, North Logan, UT (US)

Correspondence Address: UTAH STATE UNIVERSITY;TECHNOLOGY COMMERCIALIZATION OFFICE, 570 RESEARCH PARK WAY, SUITE 101, NORTH LOGAN, UT, 84341, US

(73) Assignee: Utah State University, North Logan, UT (US)

(21) Appl. No.: 12/042,495

(22) Filed: Mar. 5, 2008

Related U.S. Application Data
(60) Provisional application No. 60/893,614, filed on Mar. 7, 2007.

(51) Int. Cl. C12N 15/89; C12N 5/02; C12N 5/10

(52) U.S. Cl. 800/24; 435/377; 435/325

(57) ABSTRACT

Disclosed herein are methods of generating a recipient cytoplast for nuclear cloning with improved developmental capacity. These methods involve the in vitro maturation of an oocyte from a non-human mammal to the pre-MII stage and treatment of the oocyte with a microtubule-inhibiting agent prior to enucleation. The oocyte used to generate the recipient cytoplast can be obtained from a pre-adult or adult non-human mammal. The methods further provide for utilizing the generated recipient cytoplast for nuclear transfer, such as deriving a nuclear transfer embryo or offspring.

### RELATED APPLICATIONS

This application claims priority to U.S. Provisional Application No. 60/893,614 entitled, “COLCEMID-TREATMENT OF OOCYTES TO ENHANCE NUCLEAR TRANSFER CLONING,” which is hereby incorporated by reference in its entirety.

### FIELD

This invention relates to methods of preparing a recipient cytoplast from oocytes and further relates to methods of obtaining live offspring via nuclear transfer.

### BACKGROUND

Nuclear transfer (NT) has been successfully utilized to produce cloned offspring in a number of mammalian species, including sheep, cattle, pigs, goats, and mice. Despite these successes, the process is very inefficient, with only a portion of the clones developing to the blastocyst stage in vitro, and only a portion of those blastocysts surviving to term following implantation in a host animal. Numerous variables may be involved in the high frequency of developmental arrest and abortion of NT embryos. These include the source and quality of the recipient oocyte, the donor cell origin and cell cycle stage, the oocyte enucleation method, the NT procedure, oocyte and/or NT embryo culture conditions, and inadequate placentation. Many studies have focused on the treatment of donor cells, such as tissue origin, developmental stage (adult, fetal or embryonic), cell cycle stage, and cell lines.

In contrast to donor cells, the choice of the recipient oocyte is limited. Oocytes, obtained from pre-adult or adult animals, are either matured in vitro or in vivo, or synchronized by dibutyryl cAMP. The oocyte cytoplasm is unique, with a very delicate and complex molecular system that coordinates two haploid nuclei (from sperm and oocyte) coming together, and then supports zygotic nuclear development to term. In the context of NT, the oocyte cytoplasm can reprogram a fused somatic cell nucleus and support the subsequent development of the reconstructed oocyte to term. The cytoplast contains not only the parental genetic materials, but also many identified and unidentified elements, which sufficiently support early development of a newly created zygote. Donor nuclei are reprogrammed functionally in only a few NT embryos that develop into normal offspring. Successful reprogramming of the donor nuclei is dependent upon factors present in sufficient amounts in the recipient oocyte. Thus far very little is known about the biochemistry and physiology of an oocyte. Any cytoplasmic changes such as nuclear maturation status, changes in microtubule, microfilaments, or biochemical or physiological changes could potentially affect nuclear reprogramming and result in cloning failure.

Metaphase II (MII) oocytes have routinely been used as recipients in NT methods. However, metaphase I (MI) oocytes were shown to yield more advanced tadpole development from somatic cell NT than MII oocytes, although none developed to adulthood (DiBerardino and Hoffner, Science 219:862-864, 1983). In contrast, the rate of blastocyst development of porcine NT embryos generated using MI oocytes was significantly lower than those generated using MII oocytes (Miyoshi et al., BMC Dev. Biol. 1:12-22, 2001). The identification and selection of more suitable recipient oocyte cytoplasts would increase the success of NT.

### SUMMARY

A method of generating a recipient cytoplast for nuclear transfer cloning is disclosed that provides improved rates of blastocyst development and production of cloned offspring. This method allows the more efficient use of available immature oocytes for NT cloning and a higher rate of efficiency in obtaining NT embryos and offspring.

In a specific embodiment, the method involves in vitro maturing an oocyte obtained from a non-human mammal, culturing the matured oocyte with a microtubule (MT)-inhibiting agent, and enucleating the oocyte. The oocyte is one that has not yet reached metaphase II (pre-MII), thus this method allows the use of a larger pool of oocytes for NT cloning than has been previously available.

Oocytes collected from pre-adult or adult non-human mammals can be used to generate a recipient cytoplast in this method. Following a period of in vitro maturation, oocytes can be used to generate recipient cytoplasts regardless of the presence or absence of the first polar body (PB1).

In another embodiment, the method further involves using the recipient cytoplast to generate a NT embryo by transferring a donor cell into the space surrounding the recipient cytoplast, fusing the donor cell and cytoplast, and activating the resulting cell to begin cell division. The NT embryo can be cultured in vitro and the embryo can further be transferred to a synchronized host animal for further development.

One advantage of the method is that the resulting NT embryos can be cultured under conditions of high oxygen tension. Thus, in certain examples, NT embryos can be cultured in 5% CO₂ in air, which is an approximately 20% O₂ atmosphere.

In a specific embodiment, following culture of the NT embryo to the morula or blastocyst stage, the embryo is transferred to a synchronized host animal for in vivo development.

The foregoing and other features and advantages will become more apparent from the following detailed description, which proceeds with reference to the accompanying figures.

### BRIEF DESCRIPTION OF THE FIGURES

FIG. 1 is a diagram showing pregnancy rates throughout gestation following transfer of NT embryos derived from colcemid-treated versus untreated heifer cytoplasts. The shaded bars show pregnancy rates for NT embryos generated from MI heifer oocytes which were matured for 16-17 hours, then treated with 0.2-0.4 μg/ml colcemid for 2-3 hours. Six to seven days after NT, morulae and blastocysts were transferred to recipient cows. The solid bars show pregnancy rates for nuclear transfer embryos generated from heifer oocytes which were not treated with colcemid. Values with different superscripts (a, b) within each time point are significantly different (p<0.025).

FIG. 2 is a diagram showing MAP Kinase activity in oocytes cultured in the presence or absence of colcemid following 15 hours of in vitro maturation. Data are expressed in terms of the fold strength of kinase activity in oocytes matured for 15 hours. The values given are the mean ±SEM (n=3).

FIG. 3 is a diagram showing relative MPF activity in oocytes cultured in the presence or absence of colcemid following 15 hours of in vitro maturation. Data are expressed in terms of the fold strength of MPF activity of oocytes matured for 15 hours. Values are the mean ±SEM (n=3). Different superscripts indicate significant differences.

FIG. 4 is a diagram of the effect of electroporation with inositol 1,4,5-trisphosphate (IP₃) on the intracellular calcium release of bovine oocytes matured with or without the presence of colcemid for zero and three hours.

FIG. 5 is a diagram of the effect of electroporation with IP₃ on the intracellular calcium release of bovine oocytes matured with or without the presence of colcemid for five hours.

FIG. 6 is a diagram of the effect of electroporation with IP₃ on the intracellular calcium release of bovine oocytes matured with or without the presence of colcemid for seven hours.

### DETAILED DESCRIPTION

### I. Abbreviations

BCR: 0.3% fatty acid-free bovine serum albumin-containing CR1aa medium
BSA: bovine serum albumin
COC: cumulus-oocyte complex
FBS: fetal bovine serum
IP₃: inositol 1,4,5-trisphosphate
IVF: in vitro fertilization
MAPK: MAP kinase
MI: metaphase I
MII: metaphase II
MPF: maturation promoting factor
MT: microtubule
NT: nuclear transfer
PB1: first polar body
SCR: serum-containing CR1aa medium

### II. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “including a nucleic acid” encompasses single or plural nucleic acids, and is considered equivalent to the phrase “including at least one nucleic acid.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. For example, the phrase “mutations or polymorphisms” or “one or more mutations or polymorphisms” means a mutation, a polymorphism, or combinations thereof, wherein “a” can refer to more than one.

Although methods and materials similar or equivalent to those described herein can be used in the practice or testing of the present disclosure, suitable methods and materials are described below. The materials, methods, and examples are illustrative only and not intended to be limiting. All publications, patent applications, patents, and other references mentioned herein are incorporated by reference in their entirety.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Activating/activation: In this context, “activating” or “activation” means stimulating cell division in an ovum by fertilization or artificial means. During the process of nuclear transfer, once the enucleated recipient oocyte and the donor cell are fused, cell division is stimulated by artificial means, for example by electrical pulse, treatment with ethanol, or by treatment with a calcium ionophore, followed by addition of a protein synthesis inhibitor.

Adult: A fully grown and physically mature individual. An adult animal is one that has reached sexual maturity.

Blastocyst: The stage of embryonic development at about the 64-cell stage. A blastocyst contains outer cells (the trophoblast cells) that become the chorion, a fluid-filled cavity, and an inner cell mass that becomes the amnion and the embryo. A hatched blastocyst is one that has escaped from the zona pellucida of the oocyte and is able to implant in the uterine wall.

Chemically defined medium: Culture medium that contains a known chemical composition, in contrast to culture medium that contains fetal bovine serum (FBS).

Cloned: Cloning is the creation of a living animal/organism that is genetically essentially identical to the unit or individual from which it was produced. Cloning can use the technique of nuclear transfer, whereby a recipient enucleated oocyte is fused with a donor cell, activated to begin cell division, and cultured in vitro. Following in vitro culture, the resulting NT embryo can be transferred to a host animal for further development in vivo. A cloned animal may be at the pre-blastocyst stage, the blastocyst stage, embryonic, fetal, or a live-born individual.

Colcemid: A compound with the chemical formula C₂₁H₂₅NO₅, also known as N-deacetyl-N-methylcolchicine or demecolcine. Colcemid is a microtubule-inhibiting agent which depolymerizes microtubules, usually resulting in cell-cycle arrest at metaphase.

Cytoplast: The intact cytoplasm of a single cell that remains following enucleation. For example, the remaining cytoplasm of a cell following removal of the nuclear material. A cytoplast generated by enucleation of an oocyte can be used as a recipient for genetic material from a donor cell to generate a NT embryo.

Donor cell: A cell that provides the genetic material for cloning by NT. The donor cell can be fused with an enucleated oocyte to generate a NT embryo. Donor cells can be cells from an embryo, fetus, pre-adult, or adult animal.

Embryonic cell: A cell derived from an embryonic animal. An embryo is an organism in the early stages of growth and differentiation that is characterized by cleavage, the laying down of fundamental tissues, and the formation of primitive organs and organ systems. The embryonic stage begins at implantation in the uterus. A fertilized egg that has begun cell division, but has not yet implanted, is a pre-embryo. In the context of this application, “embryo” or “embryonic” is understood to refer to an embryo or pre-embryo.

Enucleating/enucleation: Removal of the nuclear material from a cell, while leaving the cytoplasm relatively intact. For example, removal of the nucleus from an oocyte, while maintaining the ooplasm, such that it may be used for subsequent NT cloning. Methods of enucleation include mechanical methods, chemically-assisted methods, and other methods known in the art.

Fatty acid-free bovine serum albumin: Bovine serum albumin (BSA) makes up approximately 60% of all proteins in bovine serum. It is commonly used in cell culture protocols, particularly when protein supplementation is necessary and the other components of serum are unwanted. In cell culture its main role is as a carrier of small molecules. Fatty acid-free BSA is prepared by removing the native lipids from the albumin. It is often used to deliver fatty acids and other lipids that are present in cell culture media.

Fetal cell: A cell derived from a fetal animal. A fetus is an unborn or unhatched organism in later stages of growth and differentiation, having the basic structural resemblance to the adult animal, especially after the appearance of the first bone cells.

Fused oocyte: The reconstructed cell resulting from the mechanical insertion of a donor cell into the perivitelline space of an enucleated recipient cytoplast and fusion of the membranes of the two cells.

Fused/fusing: In this context, the process by which the cell membranes of the donor cell and the recipient enucleated oocyte are combined to form a new cell. Fusion is accomplished by treating the oocyte-donor cell complex with an electrical pulse.

High oxygen tension: Culture conditions in which the atmosphere contains about at least 20% O₂.

Host animal: A surrogate animal that will gestate the developing NT embryo. For example, cattle NT embryos can be transferred to a cow that has been synchronized ±1 day to the developmental stage of the embryo. Many methods are recognized for synchronizing the reproductive cycles of animals; representative but non-limiting examples are provided herein. The resulting pregnancy in the host can be followed by ultrasound or palpation.

In vitro culture: The growth of cells outside of a living organism. Cells can be cultured on a support, such as a glass or plastic dish, or grown as a suspension. A cell culture medium is used to provide nutrients to the cells. The medium can include buffers, salts, glucose, amino acids, vitamins, and other molecules. Cell culture medium can contain animal serum, such as FBS. An example of a cell culture medium is CR1aa (Rosenkrans & First, J Anim. Sci. 72(2):434-437, 1994). Chemically-defined medium does not contain serum, but can contain proteins such as growth factors or BSA.

In vitro maturation (IVM): Culture of oocytes in vitro under conditions that promote competence for NT cloning. IVM is useful to “mature” the oocytes to the metaphase II stage. This process normally occurs within the follicle of the ovary. However, because in procedures described herein oocytes have been aspirated from “immature” follicles, they need to “mature” in vitro for a period before they gain the ability to successfully fertilize and develop. The period of IVM required prior to use of an oocyte for NT cloning varies by species and is known in the art. By way of example, the IVM period for pig oocytes is about 44 hours; for sheep oocytes, about 24 hours, and for mouse oocytes, about 16-17 hours.

MI oocyte: An oocyte that has not formed the first polar body.

MII oocyte: An oocyte that has formed the first polar body, but has not yet expelled the second polar body and is arrested at the metaphase II stage.

Microtubule-inhibiting agent: Compounds that bind to and cause depolymerization of microtubules, usually leading to cell cycle arrest at metaphase. Examples of microtubule-inhibiting agents include, but are not limited to colchicine, colcemid, vincristine, vinblastine, and nocodazole.

Morula: The stage of mammalian embryo development consisting of about 16 cells, having a small group of internal cells surrounded by a group of external cells.

Non-human mammal: Any mammal which is not a human, including, but not limited to, cattle, sheep, goats, pigs, horses, mice, rats, non-human primates, rabbits, cats, and dogs.

Nuclear transfer (NT): For the purposes of this discussion, nuclear transfer (NT) means fusion of nuclear material (e.g., an isolated nucleus or an entire cell) of a donor cell with an enucleated oocyte so that it is reprogrammed to function like a fertilized embryo. This technique is known in the art, and details can be found for instance in the following publications: Stice et al., Theriogenology 49:129-138, 1998; Solter, Nature 394:315-316, 1998; Wakayama et al., Nature 394, 369-374, 1998; Wells et al., Biol. Reprod. 57:385-393, 1997; Wilmut et al., Nature 385:810-813, 1997. In particular, nuclear transfer has been used, with moderate success, to produce clonal cattle (see, Lanza et al., Science 288:665-669, 2000; Wells et al., Biol. Reprod. 60:996-1005, 1999; Kato et al., Science 282:2095-2098, 1998; Zakhartchenko et al., Mol. Reprod. Dev. 54:264-272, 1999; Zakhartchenko et al., J. Reprod. Fert. 115:325-331, 1999; Kato et al., Science, 282:2095-2098, 1998; Lanza et al., Science, 288:665-669.)

Nuclear transfer embryo: For the purposes of this discussion a “nuclear transfer embryo” is a pre-embryo or embryo that is generated by the process of NT. An oocyte and an injected donor cell become a NT embryo when the donor cell is fused with the oocyte.

Offspring: The progeny of an animal. This includes live-born young that are not the genetic progeny of an animal, but that are the result of transfer of in vitro fertilization embryos derived from standard IVF procedures or nuclear transfer.

Oocyte: The female germ cell. An oocyte can be at any stage of oogenesis. An oocyte in the stage of meiosis I is considered a primary oocyte. An oocyte in the stage of meiosis II is considered a secondary oocyte. Once meiosis II is completed, the germ cell is no longer an oocyte, but is a mature ovum.

Perivitelline space: The space between the vitelline (plasma) membrane of an oocyte and the zona pellucida.

Polar body: The product of unequal cytokinesis in the meiotic divisions of an oocyte. When the primary oocyte divides, its nucleus, called the germinal vesicle, breaks down, and the metaphase spindle migrates to the periphery of the cell. At telophase of meiosis I, one of the two daughter cells contains hardly any cytoplasm, whereas the other cell has nearly the entire volume of cellular constituents. The smaller cell is called the first polar body, and the larger cell is referred to as the secondary oocyte. Once PB1 is ejected, the oocyte is considered a “MII” oocyte. During meiosis II, a similar unequal cytokinesis takes place. Most of the cytoplasm is retained by the mature egg (ovum), and the second polar body receives little more than a haploid nucleus.

Pre-adult: An animal that has not yet reached sexual maturity, for example, a heifer. The oocytes in a pre-adult animal remain arrested at prophase I of the first meiotic division of oogenesis.

Pre-Metaphase II oocyte: An oocyte that has not yet reached metaphase II in oogenesis. During oogenesis, a primary oocyte undergoes two meiotic divisions. The first meiotic division consists of DNA replication, prophase I, pairing of homologous chromosomes (metaphase I), anaphase I, telophase I, and cytokinesis. The first meiotic division begins during embryonic development and arrests at prophase I until the female becomes sexually mature. At the time of ovulation, an oocyte can complete meiosis I and expel the first polar body. The oocyte subsequently undergoes meiosis II, which unlike meiosis I or mitosis, does not include DNA synthesis. Instead, the oocyte undergoes prophase II, metaphase II, anaphase II, and telophase II. The oocyte then undergoes cytokinesis and expels the second polar body, becoming the mature haploid ovum.

Somatic cell: Any cell of a multi-cellular organism that will not contribute to the production of gametes. Somatic cells may be derived from animals at any stage of development, including embryonic, fetal, pre-adult, or adult.

### III. Overview of Specific Embodiments

Provided herein are methods for generation of a recipient cytoplast for nuclear transfer (NT) using a pre-metaphase II (MII) stage oocyte. These methods are based on the use of an in vitro matured oocyte from a non-human mammal that is further cultured with a microtubule-inhibiting agent prior to enucleation.

In specific embodiments, the pre-MII oocyte can be obtained from an adult or pre-adult non-human mammal, such as a cow or heifer. In a specific example, the period of in vitro maturation is a period of time during which the oocyte does not reach the MII stage. It will be recognized in the art that this period varies depending on the animal species from which the oocyte is obtained and this period can be optimized for the species utilized.

In a specific embodiment, following a period of in vitro maturation, the oocyte is treated with a microtubule-inhibiting agent. In a particular example, the microtubule-inhibiting agent can be colcemid.

A recipient cytoplast generated using pre-MII oocytes can further be utilized to generate a NT embryo. These methods include transferring donor genetic material to the oocyte, fusing the membranes of the cytoplast and donor cell, activating the fused oocyte to undergo cell division, and culturing the resulting NT embryo in vitro. In a particular embodiment, the NT embryo can be transferred to a synchronized host animal for further in vivo development.

In a specific example, the donor genetic material is an intact cell, which can be transferred to the perivitelline space of the recipient cytoplast. In a further example, the donor cell can be a somatic cell, such as a fibroblast cell. It will be recognized in the art that any diploid cell can be a suitable donor cell for NT, as can the isolated genetic material, such as a nucleus or a metaphase plate.

In further specific examples, the in vitro culture conditions of the NT embryo can be conditions of high oxygen tension. The culture medium for NT embryo in vitro culture can be chemically defined.

In a further embodiment, the NT embryo is transferred to a synchronized host animal for in vivo development. In specific examples, the NT embryo can be transferred at the morula or blastocyst stage.

Details of specific aspects of methods to generate an improved recipient cytoplast for NT cloning and further use of the cytoplast to generate NT embryos and offspring are provided below. It will be recognized that the discussion below is intended to provide representative examples and is not limiting.

### IV. Oocytes

The disclosed method utilizes pre-MII oocytes as the source for the recipient cytoplast for NT. Routinely, oocytes for use as recipient cytoplasts for NT cloning have been matured in vitro to the MII stage prior to enucleation and/or donor cell transfer. MI stage oocytes have been successfully used to generate cloned tadpoles, but not adult animals (DiBerardino and Hoffner). Attempts to use pre-MII oocytes for NT cloning have shown a lower rate of blastocyst formation than when MII oocytes are used, for example in pigs (Miyoshi et al.) and mice (Gao et al. Biol. Reprod. 67:928-934, 2002). Collected oocytes vary in physiologic status which leads to asynchronous maturation. Oocytes that have not reached MII (that is those that lack a PB1) during IVM are generally discarded. In contrast, the disclosed methods can successfully use pre-MII oocytes for NT cloning, allowing a more efficient use of oocyte resources. In a specific example, bovine pre-MII oocytes, such as those that lack the PB1, can be used to generate a recipient cytoplast for NT cloning.

Pre-MII oocytes can be obtained by dissection of follicles from ovaries of non-human mammals, for example 3-8 mm follicles with a compact and homogenous ooplasm. In one example, the pre-MII oocytes can be obtained cattle.

The disclosed method can be used with oocytes collected from adult or pre-adult non-human mammals. The developmental capacity of oocytes derived from pre-adult animals is lower than that of those derived from adult animals. See, for instance, Revel et al. (J. Reprod. Fertil. 103(1), 115-120, 1995) and Rizos et al., Theriogenology 63 (3), 939-949, 2005). The disclosed methods can utilize oocytes from pre-adult animals (such as heifers) with improved efficiency as compared to previous methods.

A. In vitro Maturation (IVM)

Oocytes used to generate a recipient cytoplast for somatic cell NT in the disclosed methods are in vitro matured for a period of time. IVM can be accomplished by in vitro culture of an immature oocyte in a cell culture medium. In one example, bovine pre-MII oocytes are cultured in TCM-199 supplemented with Earle's salts, L-glutamine, sodium bicarbonate, 10% FBS, 25 μg/ml gentamycin, 0.01 U/ml follicle stimulating hormone, and 0.01 U/ml luteinizing hormone. Other cell culture media can be used, such as NCSU-23, KSOM, or Waymouths. Pre-MII oocytes are matured in vitro for at least 12 hours (such as 12-25 hours, for example 14 hours).

B. Treatment with a MT-Inhibiting Agent

Following a period of IVM, the pre-MII oocyte can be treated with a MT-inhibiting agent. In one example, the MT-inhibiting agent is colcemid. Other MT-inhibiting agents include, but are not limited to, colchicine, colcemid, vincristine, vinblastine, and nocodazole.

The MT-inhibiting agent treatment is for a period of at least one hour, such as 2-6 hours. In one example, the oocyte is treated with at least 0.1 μg/ml colcemid in the maturation medium, such as 0.1-0.6 μg/ml colcemid.

Once treatment with a MT-inhibiting agent (e.g., colcemid) was completed, the oocytes were immediately moved into manipulation drops that also contained the same level of agent (e.g., colcemid) during the nuclear transfer process. By way of example, the transfer process in some procedures takes approximately 20 to 40 minutes per group. The oocytes then were removed from MT-inhibiting agent-containing media and processed as described herein.

### C. Oocyte Enucleation

Following IVM, the genetic material of the oocyte is removed to generate a recipient cytoplast for NT. Multiple methods of enucleation are known in the art. Mechanical methods of enucleating oocytes include, but are not limited to, mechanical aspiration of the PB1 and adjacent ooplasm, mechanical removal of the metaphase spindle under a fluorescent microscope following chromosome staining with a fluorescent dye, mechanically extruding the first polar body and surrounding cytoplasm through the zona pellucida, and aspiration of the second polar body and surrounding cytoplasm after oocyte activation. Chemically-assisted methods of enucleating oocytes include, but are not limited to, treatment with etoposide and cycloheximide to induce expulsion of the entire chromatin, treatment of MII oocytes with colcemid to create a membrane protrusion containing the chromatin, and sucrose treatment to assist with metaphase spindle visualization. Oocytes may also be enucleated by bisection of oocytes and removal of the portion containing the chromatin, or by centrifugation through a discontinuous density gradient containing cytochalasin B. (See Li et al. Cloning Stem Cells 6:3-11, 2004). Enucleation of the oocyte can be before or after transfer of donor genetic material and before or after activation. Methods of enucleating an oocyte following transfer of donor genetic material are known in the art.

In a particular example, the oocyte is enucleated before donor cell transfer by mechanical aspiration of the nuclear material. For example, the oocyte can be transferred to medium containing 5 μg/ml cytochalasin B and the nuclear material can be removed by a micropipette. The oocyte can contain the PB1, and both the PB1 and nuclear material can be removed by a micropipette.

### V. Donor Cells

Methods are provided for further utilizing the disclosed improved recipient cytoplast to generate a cloned NT embryo or offspring. A donor cell is used to provide the diploid genetic material to reconstitute the enucleated oocyte. An intact donor cell or the genetic material isolated from a donor cell can be used. If an intact donor cell is used, the whole cell can be transferred by a micropipette to the perivitelline space of the recipient cytoplast. If isolated genetic material from a donor cell (such as a nucleus or metaphase plate) is used, the genetic material is introduced directly into the oocyte.

The donor cell can be differentiated or undifferentiated, such as cells from an embryo, fetus, pre-adult, or adult non-human mammal. Cells for use as donor cells can be obtained by methods known in the art. Non-human mammalian cells useful as donor cells can include, but are not limited to, somatic cells such as fibroblasts, keratinocytes, myocytes, cumulus cells, endothelial cells, epithelial cells, melanocytes, hematopoietic cells, and mammary gland cells. Germ cells obtained from an embryo, fetus or adult can also be used as donor cells. A donor cell can be taken from a non-human mammal with desirable characteristics. A donor cell can also be a cell from a genetically modified non-human mammal, such as a transgenic animal.

The source of the donor cells is not an essential aspect of the disclosed invention. In a particular example, the donor cell is a fibroblast cell. The cell can be derived from a primary culture, for example one established from an skin biopsy. Donor cells can be quiescent or proliferating. Donor cells can also be arrested at a particular stage of the cell cycle, such as metaphase or G1 phase, by methods known in the art. In one example, actively dividing cells in culture are used as donor cells.

### VI. Fusion and Activation

To generate a NT embryo, the membranes of the oocyte and donor cell must be fused and the resulting fused oocyte must be activated to begin cell division.

### A. Fusion

If an intact cell is used as the donor genetic material, the cell membranes of the recipient cytoplast and the donor cell are fused. Fusion can be accomplished by treatment with an electrical pulse. In a particular example, the pulse is direct current of 1.2 kV/cm for 25 μsec in a solution of 0.27 M mannitol, 0.1 mM CaCl₂, 0.1 mM MgCl₂, and 0.05% BSA.

If isolated genetic material from a donor cell is used, the fusion step is not necessary. Thus, another option is direct injection of a nucleus into the cytoplast. This is a common practice in the mouse and primates, and can be used in other species as well.

### B. Activation

The oocyte or the fused oocyte must be activated to begin cell division. An oocyte can be activated before enucleation and/or before the introduction of the donor cell or genetic material. The oocyte can also be activated after enucleation and/or after the introduction of the donor cell and fusion. By way of example, fused oocytes may be incubated for 2 to 4 hours in CR1aa under standard culture conditions before activation.

Fused oocytes can be activated by methods known in the art, such as treatment with a calcium ionophore, ethanol, direct current pulses, or injection of fertilized oocyte cytoplasm. In one example a calcium ionophore (such as ionomycin, A23817 or other ionophores) can be used to activate a fused oocyte. In a particular example, a fused oocyte can be activated by treatment with about 1-100 μM ionomycin (such as 5 μM ionomycin) for 1-15 minutes (such as 5 minutes).

The activation process can include agents that inhibit protein synthesis and/or MT-inhibiting agents. Protein synthesis inhibitors include cycloheximide or DMAP (6-dimethylaminopurine). For example, the protein synthesis inhibitor cycloheximide can be added to the culture medium following an activation step at about 1-100 μg/ml cycloheximide (such as 10 μg/ml cycloheximide) for 1-10 hours (such as 5 hours). Activated oocytes can also be treated with MT-inhibiting agents, such as cytochalasin B or nocodazole. Other treatment methods, such as the inclusion of protein kinase inhibitors are known in the art.

VII. In vitro Culture of NT Embryos

Following activation, resulting NT embryos can be cultured in vitro for further development. NT embryos can be cultured in an appropriate cell culture medium, such as CR1aa, KSOM, NCSU-23, BARC, or G1.2/G2.2. These culture media can be supplemented with fetal bovine serum (FBS), can be of a chemically defined composition, or can be conditioned by previous culture with other cells. The NT embryos can also be co-cultured with other cells to mimic the in vivo environment.

In a particular example, NT embryos can be co-cultured with a monolayer of cumulus cells in CR1aa medium supplemented with FBS. In another example, NT embryos can be cultured in the absence of co-culture. In a further example, NT embryos can be cultured in a chemically defined medium, such as CR1aa supplemented with fatty acid-free BSA.

Oxygen tension has been reported to play a role in successful development of NT embryos during in vitro culture. Reduced oxygen tension has been shown to increase development to the blastocyst stage compared with high oxygen tension (Olson and Seidel, J. Anim. Sci. 78:152-157, 2000; Dumoulin et al. Hum. Reprod. 14:465-469, 1999). NT embryos obtained by the disclosed methods can be successfully cultured in an atmosphere of increased oxygen. In a particular example, NT embryos derived from colcemid-treated oocytes are cultured under conditions of high oxygen tension (such as about 20% O₂, for example 5% CO₂ in air).

The development of NT embryos can be monitored at various time points during in vitro culture. The number of NT embryos reaching particular developmental stages can be determined, such as the two-cell stage (cleavage), morula, blastocyst, or hatched blastocyst.

The quality of the developing NT embryos can be determined by determining the number of cells present or the chromosomal composition of the cells in a NT embryo. In one example, cell number can be determined by counting the number of nuclei present in a NT embryo. In another example, chromosome composition of the NT embryo cells can be examined to determine the cell ploidy. In a particular example, NT embryos can be incubated in 0.1 μg/ml colcemid in culture medium for five hours, treated with 1% trisodium citrate for 8-10 minutes and fixed on a glass slide for chromosome examination under light microscopy.

### VIII. Transfer of NT Embryos to a Host Animal

Following in vitro culture, NT embryos can be transferred to a host animal of the same species for further in vivo development. Methods of surgical and non-surgical transfer are known in the art. In one example, NT embryos that have developed to the morula stage can be transferred to a host animal that is synchronized to the stage of the embryos. There are several known methods for synchronizing the reproductive cycles of animals. For instance, it is easier using in vitro produced embryos rather than trying to synchronize an embryo donor animal to recipient animals. Synchrony in this case is matching the developmental stage of the embryo (NT) to the stage of cycle in the recipient animal. If a large number of potential recipient animals are available, the “natural” heats of the animals can be used by selecting a set of animals that have coincident cycles. In general, 5% of the population should be in heat each day within the random population. Several types of hormonal treatments can also be employed to get animals to show estrus (“heat”; day=0) on a specific schedule. These include using (injected) prostaglandin F2-alpha, either as a single shot (dose) (which results in approximately 45% of the animals randomly receiving the shot coming into “heat” within 72 hrs), or administering two doses 11 days apart (which results in 90% of the treated animals coming into heat after the second dose). Another approach is to provide the animal with ongoing dosage of progesterone for 8 to 14 days (depending on the regime used). Once the progesterone dose is removed, the animals will then come into “heat”.

In another example, NT embryos that have developed to the blastocyst stage can be transferred to a host animal that is synchronized to the stage of the embryos. A resulting pregnancy can be determined by ultrasound, such as trans-rectal ultrasound between 25-30 days following transfer. Ongoing pregnancy can be monitored by ultrasound or palpation.

### EXAMPLES

The following examples are provided to illustrate certain particular features and/or embodiments. These examples should not be construed to limit the invention to the particular features or embodiments described.

### Example 1

### Development of NT Embryos Derived from Colcemid-Treated Cytoplasts

This example describes the effect of colcemid treatment of oocytes following in vitro maturation (IVM) on subsequent development of NT embryos.

### Methods and Materials

Oocyte Maturation in vitro (IVM)

Bovine cumulus-oocyte complexes (COC) were aspirated from 3-8 mm diameter follicles on ovaries obtained from a local slaughterhouse. Only COC with a compact and a homogenous ooplasm were selected. The COC were matured in TCM 199 with Earle's salts, L-glutamine and sodium bicarbonate (Gibco Inc., Grand Island, N.Y.) supplemented with 10% FBS (HyClone, Logan, Utah), 25 μg/ml gentamycin, 0.01 U/ml FSH (NIH-FSH-S17), 0.01 U/ml LH (USDA-bLH-6) in four-well plates with 0.5 ml medium and 30-50 oocytes/per well at 39° C. in humidified atmosphere of 5% CO₂ and air.

### Donor Cell Culture and Preparation

Primary bovine fibroblast cultures were established from ear biopsy. Tissues were washed and minced, suspended in DMEM/F12 (1:1) supplemented with 15% FBS and antibiotics, seeded in 25 cm² flasks, and cultured at 37° C. in a humidified atmosphere of 5% CO₂ and air for several days. The cells were passaged when they reached over 80% confluence. Cells from passages 2-13 were used for NT. Immediately before NT, the cells at 80% confluence were dissociated by trypsinization with 0.25% trypsin with EDTA solution (HyClone), without previous serum starvation or medium change during the culture period. Small-size (10-12 μm) cells with a round shape were used as donor cells.

Treatment of Recipient Oocytes with Colcemid

Oocytes matured for 14-15 hr, 16-17 hr, or 19-20 hr, respectively, were used as recipients for nuclear transfer in this study. Cumulus cells were removed by vortexing COC in TL-Hepes medium containing 100 IU/ml hyaluronidase. Oocytes with PB1 and without PB1 were cultured in maturation medium with 0.1 μg/ml-0.4 μg/ml colcemid for 1-4 hours.

### Enucleation and Nuclear Transfer

After treatment with colcemid, oocytes with a protruding vitelline membrane were transferred into TL-Hepes medium containing 5.0 μg/ml cytochalasin B and the protrusion and first polar body were removed by pipette. If there was no PB1, the membrane protrusion was simply removed. Single donor cells were individually transferred to the perivitelline space of the recipient cytoplasts, forming a couplet.

### Fusion and Activation

The couplets were fused using one direct current pulse of 1.2 kV/cm for 25 μsec by an Electro Cell Manipulator 2001 (BTX, San Diego, Calif.) in 0.27 M mannitol, 0.1 mM CaCl₂, 0.1 mM MgCl₂, and 0.05% BSA. Fused oocytes were activated at 24 to 25 hours (initiation of IVM=0 hr) with 5 μM ionomycin for 5 minutes, followed by treatment with 10 μg/ml cycloheximide in CR1aa plus 3% FBS for 5 hours at 39° C. in 5% CO₂ in air. The interval between fusion and activation was 2 to 4 hours.

### In Vitro Culture of NT Embryos

After activation, NT embryos were cultured under mineral oil in 30 μl droplets of CR1aa+3% FBS with a monolayer of bovine cumulus cells at 39° C. in 5% CO₂ and air for 7 days (activation as Day 0). Cleavage, morula and blastocyst development were evaluated at 48 hr, Day 5 and Day 7 after activation, respectively.

### Chromosomal Analysis and Cell Number Count

Day 7 blastocysts were prepared and examined for their cytogenetic composition and the number of nuclei contained was verified. Briefly, blastocysts were incubated in 0.1 μg/ml of colcemid (Gibco) in culture medium for 5 hr., treated in 1% trisodium citrate for 8-10 min and fixed on clean glass slides. Chromosomes were examined at 1000× under a Zeiss microscope, and the chromosome composition was determined for each blastocyst. Any embryo that did not show an interpretable metaphase spread due to gross overspreading or clumped chromosomes was excluded.

### Results

Treatment of Oocytes with Colcemid

In the described IVM system, approximately 9%, 30%, and 75% of bovine oocytes matured for 14-15 hr, 16-17 hr, and 19-20 hr expel a PB1, respectively. The majority of PB1-free oocytes are at metaphase I stage. Of the oocytes matured for 16-17 hr, approximately 80% and 80-93%, respectively, of the oocytes with or without PB1 exhibited an obvious membrane projection after colcemid treatment for 2 to 4 hr (Table 1). Similar results were observed with oocytes matured for 14-15 hr and 19-20 hr.

TABLE 1
Membrane projections of bovine oocytes matured for 16-17 hr
and then treated with different concentrations of colcemid and
for different lengths of time.

| Oocytes with (+) | | | No. oocytes with |
|---|---|---|---|
| or without (−) | Colcemid | No. oocytes | blebs or Pb1 + bleb |
| PB1 | treatment | examined | (%) |
| − | 0.1 μg/ml for 1 h | 46 | 32 (69.6) |
| − | 0.1 μg/ml for 2 h | 48 | 39 (81.2) |
| − | 0.1 μg/ml for 4 h | 46 | 43 (93.4) |
| − | 0.2 μg/ml for 2 h | 42 | 33 (78.6) |
| − | 0.2 μg/ml for 4 h | 42 | 38 (90.5) |
| − | 0.4 μg/ml for 2 h | 43 | 35 (81.3) |
| − | 0.4 μg/ml for 4 h | 41 | 34 (82.9) |
| + | 0.2 μg/ml for 2 h | 36 | 26 (72.2) |
| + | 0.2 μg/ml for 4 h | 39 | 31 (79.5) |
| + | 0.4 μg/ml for 2 h | 38 | 29 (76.3) |
| + | 0.4 μg/ml for 4 h | 38 | 28 (73.6) |

Development of NT Embryos Derived from Colcemid-Treated Cytoplasts

There was no difference in cleavage among the different treatment groups and development was similar to the controls (Table 2). After the oocytes matured for 14-15 hr the majority of them had expelled PB1. The treated PB1-free oocytes resulted in 40% blastocyst development. Within the oocyte group matured for 16-17 hr, blastocyst development was 44.4% and 42.2%, respectively, of treated oocytes with or without PB1, and was higher (P<0.05) than the control (29.8%). In the group of oocytes matured for 19-20 hr, the treated PB1-free oocytes resulted in 38% blastocyst development, which was significantly higher than the treated PB1 oocytes and the control. Cell numbers of blastocysts in colcemid-treated groups were numerically higher than the control groups. The majority (75%-86%) of the chromosomal composition of the cloned blastocysts was diploid both in the colcemid-treated and in the control groups.

TABLE 2
Development of NT embryos after the recipient oocytes were treated with 0.2 μg/ml
colcemid for 2-3 hr

| | | | | | | | |
|---|---|---|---|---|---|---|---|
| | | | | | | Cell | % |
| | No. | | | | Hatching | number of | Diploid |
| Treatment | embryos | 2-cell | Morula | Blastocyst | Blastocyst | blastocysts | blastocyst |
| groups | cultured | (%) | (%) | (%) | (%) | (mean ± SD) | (n) |

Oocytes matured for 14-15 hr

| | | | | | | | |
|---|---|---|---|---|---|---|---|
| Oocytes | 100 | 85 (85) | 48 (48) | 40 (40)^{a} | 26 (26)^{a} | 113 ± 10 | 79.4 (34) |
| without | | | | | | | |
| PB1* | | | | | | | |
| Control** | 36 | 29 (80.6) | 16 (44.4) | 13 (36.1)^{a} | 5 (13.9)^{b} | 98 ± 15 | 81.2 (11) |

Oocytes matured for 16-17 hr

| | | | | | | | |
|---|---|---|---|---|---|---|---|
| Oocytes | 81 | 69 (85.2) | 45 (55.6) | 36 (44.4)^{a} | 21 (25.9)^{a} | 103 ± 20 | 75 (32) |
| with PB1* | | | | | | | |
| Oocytes | 90 | 76 (84.4) | 44 (48.9) | 38 (42.2)^{a} | 22 (24.2)^{a} | 118 ± 17 | 80.6 (31) |
| without | | | | | | | |
| PB1* | | | | | | | |
| Control** | 57 | 47 (82.5) | 20 (35.1) | 17 (29.8)^{b} | 6 (10.5)^{b} | 93 ± 23 | 82.3 (17) |

Oocytes matured for 19-20 hr

| | | | | | | | |
|---|---|---|---|---|---|---|---|
| Oocytes | 107 | 88 (82.2) | 38 (35.5) | 30 (28.0)^{b} | 6 (15.0)^{b} | 91 ± 9 | 75 (28) |
| with PB1* | | | | | | | |
| Oocytes | 66 | 56 (84.8) | 32 (48.5) | 25 (37.9)^{a} | 14 (21.2)^{a} | 107 ± 11 | 86.3 (22) |
| without | | | | | | | |
| PB1* | | | | | | | |
| Control** | 87 | 69 (79.3) | 26 (30.0) | 21 (24.1)^{b} | 7 (8.0)^{b} | 93 ± 9 | 77.8 (18) |

The same column, a, b: P < 0.05.
*Indicates the oocytes with or without PB1 at the beginning of treatment by colcemid.
**Oocytes used in control were those with PB1 from the same batch as the treated group but without colcemid treatment.

### Example 2

### Effect of Varying Colcemid Treatment Conditions on NT Embryo Development

This example describes the effects of various colcemid treatment conditions on the in vitro development of NT embryos to the blastocyst stage.

### Methods and Materials

The methods used are described in Example 1. Briefly, oocytes, regardless of PB1 status, were all treated with colcemid, and the oocytes with projections were used as recipient cytoplasts. To determine whether treatment time or concentration of colcemid affected NT embryonic development, oocytes matured for 16-17 hr were treated with 0.2 and 0.4 μg/ml colcemid for 2-3 hr and 5-6 hr, respectively.

### Results

The colcemid concentration (0.2 and 0.4 μg/ml) and treatment time (2 to 6 hr) did not affect NT embryo development to the blastocyst and hatching blastocyst stages (Table 3). The colcemid treated oocytes resulted in 39% to 42% blastocyst development which were significantly higher than the control group (30%).

TABLE 3
Development of NT embryos derived from oocytes matured for
16-17 hr and then treated with colcemid for different time periods

| | No. embryos | | Morula | Blastocyst |
|---|---|---|---|---|
| Treatment groups | cultured | 2-cell (%) | (%) | (%) |
| 0.2 μg/ml for 2-3 h | 90 | 76 (84.4) | 44 (48.9)^{a} | 38 (42.2)^{a} |
| 0.2 μg/ml for 5-6 h | 76 | 69 (90.8) | 37 (48.7)^{a} | 32 (42.1)^{a} |
| 0.4 μg/ml for 2-3 h | 73 | 66 (90.4) | 37 (50.7)^{a} | 32 (43.8)^{a} |
| 0.4 μg/ml for 5-6 h | 81 | 66 (81.5) | 39 (48.1)^{a} | 31 (38.8)^{a} |
| Control | 57 | 47 (82.5) | 20 (35.1)^{b} | 17 (29.8)^{b} |

The same column, a, b: P < 0.05.

A high rate of blastocyst development of cloned embryos is necessary for animal cloning, which provides much more availability and opportunity for both embryo transfer and basic molecular studies. These data show that colcemid-treated heifer oocytes both with and without PB1 improved the ability of cytoplast to support blastocyst development. The average blastocyst development rate was over 40% under 5% CO₂ in air, especially for those oocytes matured for 14 to 17 hr and treated with colcemid. The quality of blastocysts derived from colcemid-treated oocytes was also greatly improved over the untreated controls. For example, the rate of hatched blastocysts was much higher in the colcemid groups than the untreated groups. The cell number of the treated blastocysts was numerical higher than the controls, and the majority of the blastocysts were diploid.

### Example 3

### Culture of NT Embryos from Colcemid-Treated Oocytes in Different Systems

This example shows the effect of colcemid-treated cytoplasts on development of NT embryos in vitro cultured under different conditions. The effect of different NT embryo culture media on development is shown, as well as NT embryo development with co-culture with bovine cumulus cells.

### Methods and Materials

Collection and IVM of oocytes was as described in Example 1. Oocytes were matured for 16-17 hours, treated with 0.2 μg/ml colcemid for 2-3 hours, and used for NT as described in Example 1. NT embryos were cultured in CR1aa+3% FBS (serum-containing CR1aa; SCR) or CR1aa+0.3% fatty acid-free BSA (BSA-containing CR1aa; BCR), with or without bovine cumulus cell co-culture. Blastocyst chromosome complement and cell number were determined as described in Example 1.

### Results

After culture of the clones in SCR or BCR media the rate of morula and blastocyst development was improved in the colcemid treatment groups compared to the untreated groups (Table 4). The chemically defined medium, that is BCR, increased the morula and blastocyst development of NT embryos derived from colcemid-treated oocytes (18.6% vs. 5.6% of untreated oocytes) under 5% CO₂ in air. The highest blastocyst development (39.5%) was observed in the colcemid treatment and co-culture group.

Blastocyst cell numbers were much higher (P<0.01) in the co-culture system than in SCR and BCR culture systems. The cell number of blastocysts derived from colcemid-treated oocytes increased when embryos were cultured in SCR and BCR systems. Chromosomal analyses showed that the majority of treated and untreated blastocysts were diploid.

TABLE 4
Effect of different culture media on development of NT embryos derived from
oocytes matured for 16-17 hours and then treated with 0.2 μg/ml colcemid for 2-3
hours.

| | | No. | | | | | | |
|---|---|---|---|---|---|---|---|---|
| | | cloned | | | | Hatched | No. cells | % diploid |
| Colcemid | Culture | embryos | 2-cell | Morula | Blastocyst | blastocyst | blastocysts | blastocyst |
| treatment | system* | cultured | (%) | (%) | (%) | (%) | (mean ± SD) | (n) |
| + | SCR | 135 | 96 (71.1) | 41 (30.3)^{b} | 31 (23.0)^{b} | 5 (3.7)^{b} | 70.5 ± 7.8^{b} | 88 (25) |
| − | SCR | 125 | 90 (72) | 33 (26.4)^{b} | 20 (16)^{c} | 0 | 56.8 ± 5.6^{c} | 89.5 (19) |
| + | BCR | 110 | 81 (73.6) | 27 (24.5)^{b} | 20 (18.2)^{c} | 0 | 50.3 ± 7.6^{c} | 82.3 (17) |
| − | BCR | 107 | 66 (61.7) | 13 (12.1)^{c} | 6 (5.6)^{c} | 0 | 40.2 ± 10.5^{d} | 100 (6) |
| + | SCR and | 86 | 74 (86.0) | 42 (48.8)^{a} | 34 (39.5)^{a} | 19 (22.0)^{a} | 107.8 ± 21.6^{a} | 77.8 (27) |
| | co- | | | | | | | |
| | culture | | | | | | | |
| − | SCR and | 75 | 58 (77.3) | 22 (29.3)^{b} | 19 (25.3)^{b} | 8 (10.7)^{b} | 96.7 ± 19.4^{a} | 81.2 (16) |
| | co- | | | | | | | |
| | culture | | | | | | | |

The same column, a, b and b, c and c, d: P < 0.05; a, c and a, d and b, d: P < 0.01.
*SCR: serum-containing CR1aa; BCR: BSA-containing CR1aa; co-culture: culture in the presence of bovine cumulus cells.

### Example 4

### Development of NT Embryos Transferred to Recipient Cows

This example describes the development of NT embryos following transfer to synchronized recipient animals.

### Methods and Materials

MI stage heifer oocytes were matured for 16-17 hr then treated with 0.2 μg/ml or 0.4 μg/ml colcemid for an additional 2-3 hr as described in Example 1. The day 6-7 morulae and blastocysts were selected and transferred to recipient cows. A total of 191 recipient cows were used for embryo transfer. Forty cows received embryos from colcemid-treated oocytes and 151 cows received embryos from non-treated oocytes. Pregnancy rates were monitored throughout gestation.

### Results

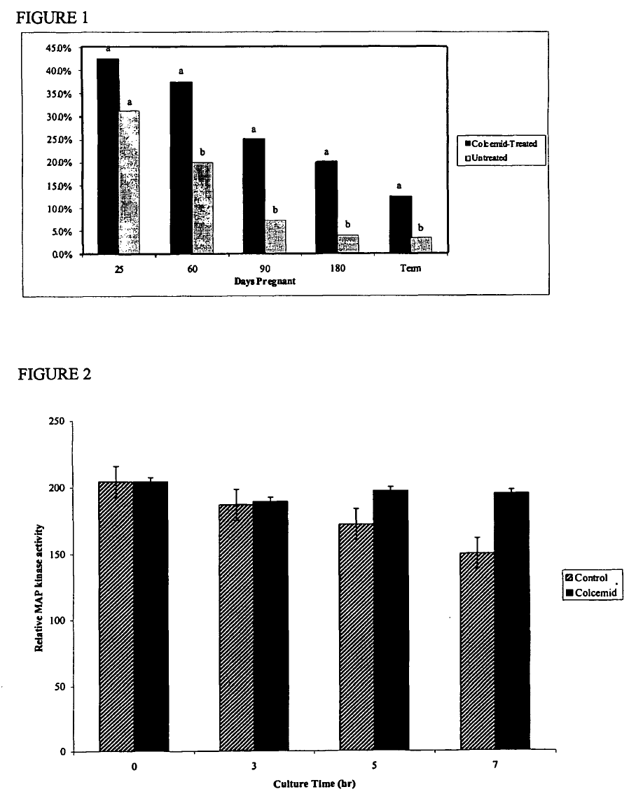

Higher (P<0.05) pregnancy rates at 60, 90, 180 days were obtained in the treated group than the non-treated group. The term rate was 12.5% of the embryos from colcemid-treated cytoplasts, which was significantly higher than that from non-treated group (3.3%) (FIG. 1). An increase from 3.3% to 12.5% in term pregnancies by treating the oocytes with colcemid prior to NT represents a significant improvement over untreated heifer oocytes. These results suggest that that the treatment of oocytes with colcemid prior to enucleation is more efficient in reprogramming the donor cell and directing development throughout gestation.

### Example 5

### Effect of Colcemid Treatment on Oocyte MAP Kinase Activity

This example describes MAP kinase (MAPK) activity of in vitro matured oocytes treated with or without colcemid. The activity of the MAPK isoform ERK2 was measured.

### Methods and Materials

### Oocyte Collection, In Vitro Maturation, and Colcemid Treatment

All reagents were purchased from ICN Biomedicals Inc. (Irvine, Calif.) unless otherwise stated. All procedures were performed according to published methods (Campbell et al. Biol. Reprod. 62:1702-1709, 2000). Bovine oocytes were collected from a local abattoir (E. A. Miller, Hyrum, Utah). Oocytes from follicles 3-8 mm in size were aspirated into 50-ml centrifuge tubes using an 18-gauge needle attached to a vacuum pump. Oocytes with uniform cytoplasm and intact multiple layers of cumulus cells were selected and washed with PB1+ (phosphate-buffered saline with Ca²⁺ and Mg²⁺ plus 5.55 mM glucose, 0.32 mM sodium pyruvate, 3 mg/ml BSA). Oocytes were transferred into 500 μl of maturation medium (M199 containing 10% FBS (Hyclone Laboratories, Logan, Utah), 0.5 μg/ml FSH (Sioux Biochemicals, Sioux City, Iowa), 5 μg/ml LH (Sioux Biochemicals), 100 units/ml penicillin (Life Technologies, Grand Island, N.Y.), and 100 μg/ml streptomycin (Life Technologies)) in four-well culture dishes (Nunc, Milwaukee, Wis.) and cultured at 39° C. in a humidified atmosphere of 5% CO₂ and air for 15 hours.

At 15 hr after the initiation of maturation, oocytes were vortexed in 1 ml PB1+ containing 10 mg/ml hyaluronidase for 4 min. to completely remove cumulus cells. Oocytes were randomly assigned to the treatment groups and transferred into 500 μl of maturation medium or 500 μl of maturation medium containing 0.4 μg/ml colcemid (KaryoMax Colcemid Solution, Gibco). Oocytes from each treatment group were removed from maturation medium and placed in double-strength sample buffer (0.5 ml 1 M Tris-HCl (pH 6.8), 0.5 ml β-mercaptoethanol, 1 ml glycerol, 2 ml 10% SDS, 1 mg bromophenol blue, and 2 ml ddH₂O) at 15, 18, 20, and 22 hours respectively.

### Western Blot Analysis of MAPK

Western blot analysis of MAPK was performed according to the procedures used by Sun and Fan (Methods Mol Biol. 253:293-304, 2004). Briefly, 25 bovine oocytes were added to 6.5 μl double-strength sample buffer and 5 μl ddH₂O in a 0.5 ml Eppendorf tube and frozen at −80° C. until use. The samples were boiled for 4 minutes, placed on ice for 5 minutes, and centrifuged at 12,000 g for 3 minutes. The total proteins were separated by SDS-page with a 4% stacking gel and a 10% separating gel for 45 minutes at 110 V and 115 mA. (Pierce Precise Protein Gels) while the nitrocellulose membrane and filters were incubated in the transfer buffer at 4° C. for 45 minutes. After electrophoresis, the gel was washed with transfer buffer for 3 minutes and then the total protein was transferred electrophoretically onto the nitrocellulose membrane for 20 minutes at 20 V. After transfer the membrane was washed twice with TBS for 5 minutes each time and then the membrane was blocked overnight at 4° C. in TBST with 5% skim milk.

To detect total ERK2 the membrane was incubated for 1 hour with polyclonal rabbit anti-ERK2 antibody diluted 1:300 in TBST, washed 3 times in TBST for 10 minutes each, incubated for 1 hour at 37° C. with HRP-labeled goat anti-rabbit IgG diluted 1:1000 in TBST, and finally processed as described below. The nitrocellulose membrane was washed two times with TBST, 5 minutes each time. For protein detection one part Opti-4CN dituent concentrate was mixed with nine parts ddH₂O (BioRad Opti-4CN Substrate Kit). 0.2 ml of Opti4CN substrate was added to 10 ml of diluent, mixed well, and poured onto the membrane. The membrane was incubated with gentle agitation in the substrate for 30 minutes and washed in ddH₂O for 15 minutes. Densiometric analysis was performed using the Scion Image software.

### Statistical Analysis

The relative activities of MAPK were compared using the General Linear Model (PROC GLM) of the Statistical Analysis Software (SAS) software.

### Results

MAP kinase activity in oocytes was determined by measurement of ERK2 activity. The results from the ANOVA analysis indicated there was evidence of differences among the relative mean MAPK activities (p<0.05). MAPK activity of control and colcemid-treated oocytes is shown in FIG. 2. The relative activity of MAP kinase was significantly different between the colcemid treated oocytes and the control oocytes (p<0.05). The length of time of maturation did not have an effect on the MAP kinase activity regardless of treatment level (p>0.05) and the interaction between time and treatment was not significant (p>0.05). The difference between MAPK activity between control and treated oocytes was significant at all time points.

### Example 6

### Maturation Promoting Factor Activity in Oocytes Treated with Colcemid

The example describes the effect of treatment of oocytes with colcemid on maturation promoting factor (MPF) activity.

### Materials and Methods

Oocyte collection, in vitro maturation, and colcemid treatment were as described in Example 5.

### MPF Activity

MPF activity was determined using a cdc2 kinase assay. Ten oocytes/treatment were added to 5 μl of sample buffer (50 mM Tris-HCl (pH 7.5), 0.5 M NaCl, 5 mM EDTA, 2 mM EGTA, 0.01% Brij35, 1 mM PMSF, 0.05 mg/ml leupeptin, 50 mM 2-mercaptoethanol, 25 mM beta glycerophosphate, 1 mM Na-orthovanadate). The samples were frozen in liquid nitrogen and sonicated for 75 seconds at 4° C. using a Branson 1200 Sonicator (Branson Ultrasonics Corp., Danbury, Conn.). The samples were stored at −80° C. until use. The MESACUP cdc2 Kinase Assay Kit (MBL, Nagoya, Japan) was used to determine the MPF activity. The cdc2 kinase assay was performed on ice according to the protocol described by MBL. 5 μl of oocyte extract was mixed with 5 μl of 10× cdc2 Reaction Buffer, 5 μl of biotinylated MV Peptide, 30 μl of double-distilled water, and 5 μl of 1 mM ATP solution. The contents of the tube were mixed well and incubated for 30 minutes at 30° C. in a water bath. The reaction was terminated by adding 200 μl of the phosphorylation Stop Reagent and centrifuged for 15 seconds at 15,000 rpm. 100 μl of the terminated reaction mixture were transferred to a microwell coated with Monoclonal Antibody (4A4). The microwells were incubated at 25° C. for 60 minutes and then washed five times with the Wash solution. 100 μl of peroxidase-conjugated streptavidin was added to each well, incubated at 25° C. for 30 minutes, and each well was washed 5 times with Wash solution. 100 μl of peroxidase substrate solution was added to each well and then incubated at 25° C. for 4 minutes, at which time 100 μl of Stop Solution (20% H₃PO₄) was added to each well. The OD of each well was read at 492 nm with a SpectraMax® Plus microplate spectrophotometer (Molecular Devices, Sunnyvale, Calif.). Data were expressed in terms of absorbance at 492 nm.

### Statistical Analysis

The relative activities of MPF were compared using the General Linear Model (PROC GLM) of the Statistical Analysis Software (SAS) software, as above.

### Results

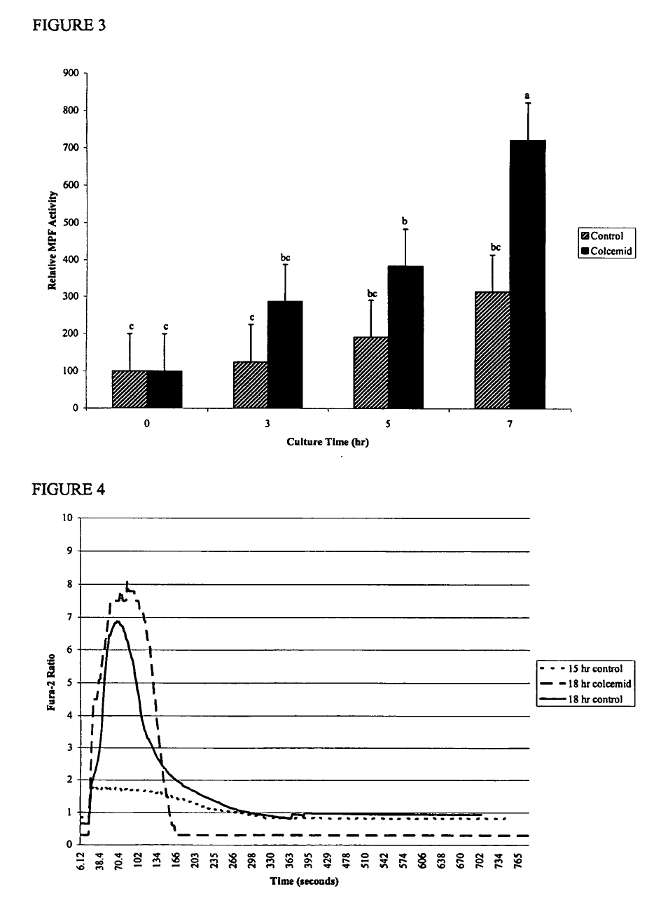

The results from the ANOVA analysis indicates there is evidence of differences among the relative mean MPF activities (p<0.0001). Relative MPF activity of oocytes treated with or without colcemid is shown in FIG. 3. The relative mean MPF activity of oocytes matured in the presence of colcemid was significantly higher than oocytes matured without colcemid (p<0.0001). The length of time in maturation also had an effect on the relative mean MPF activity (p<0.0001) with the oocytes matured for 22 hrs having significantly higher activity compared to oocytes matured for 15, 18, or 20 hrs. Also the interaction of the time and treatment variables had an effect on the relative mean MPF activity (p<0.0001). The colcemid treated oocytes matured for 22 hrs had a significantly higher MPF activity compared to every other time and treatment combination.

### Example 7

### Intracellular Calcium Transients in Oocytes Treated with Colcemid

This example describes the effect of treatment of oocytes with colcemid on the generation of intracellular calcium transients.

### Materials and Methods

Oocyte collection, in vitro maturation, and colcemid treatment were as described in Example 5.

Oocyte Loading with Calcium Indicator

Oocytes were vortexed in 1 ml PB1+ containing 10 mg/ml hyaluronidase for 4 minutes to completely remove cumulus cells. Oocytes of good quality were selected for use. Oocytes were loaded with Ca²⁺ indicator by incubation in 2 μM Fura-2 AM ester (Molecular Probes, Eugene, Oreg.) and 0.02% Pluronic F-127 (Molecular Probes) in Ca²⁺- and Mg²⁺-free phosphate buffered saline (Hyclone Laboratories) containing 0.32 mM sodium pyruvate, 5.55 mM glucose, 3 mg/ml BSA, and 100 μM EGTA at 39° C. in darkness for 45 min. After loading with indicator, oocytes were washed extensively with PB1+ (phosphate-buffered saline with Ca²⁺ and Mg²⁺ plus 5.55 mM glucose, 0.32 mM sodium pyruvate, 3 mg/ml BSA) and maintained in this solution at 39° C. until use.

### Intracellular Calcium Monitoring

Fura-2 loaded oocytes were transferred to a 50-μl drop of PB1+ medium covered with mineral oil. Intracellular Ca²⁺ monitoring and electroporation conditions were according to published methods (Campbell et al. Biol. Reprod. 62:1702-1709, 2000; Viets et al. Cloning Stem Cells 3:105-113, 2001; Yue et al. Development 121:2645-2654, 1995; Yue et al. Biol. Reprod. 58:608-614, 1998).

### Results

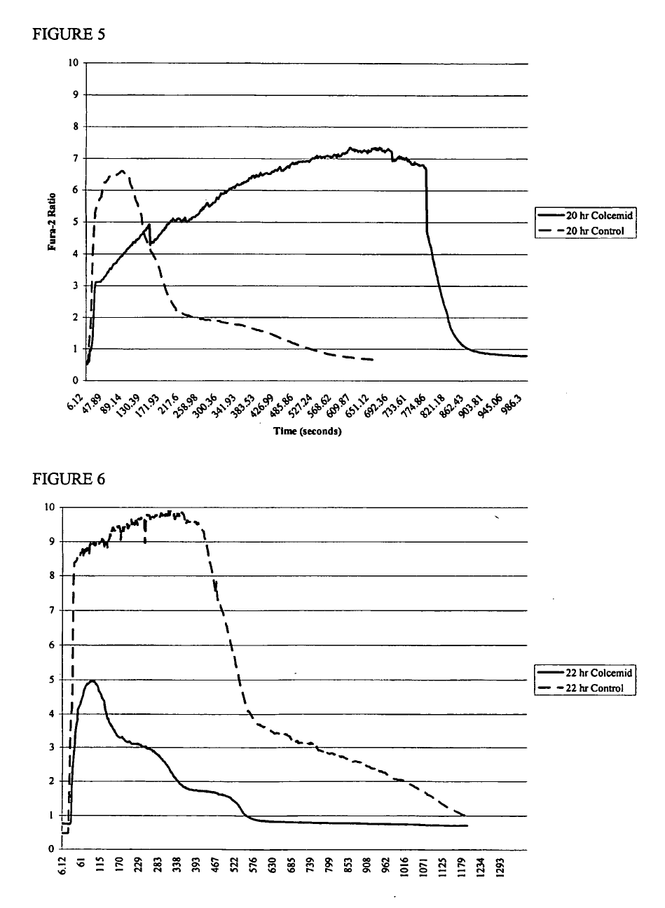

The results for the intracellular calcium transients are listed in Table 5 and representative traces are given in FIGS. 4-6. No trend between treatments could be discerned from the data.

TABLE 5
Number of oocytes responding to electroporation with
inositol 1,4,5-trisphosphate (IP₃).

| | No. of | No. of |
|---|---|---|
| | Oocytes | Oocytes |
| Treatment Group | Tested | Responding |
| 15 hr maturation | 10 | 7 |
| 18 hr maturation, 3 hr colcemid | 9 | 7 |
| 18 hr maturation, control | 10 | 8 |
| 20 hr maturation, 5 hr colcemid | 9 | 9 |
| 20 hr maturation, control | 7 | 7 |
| 22 hr maturation, 7 hr colcemid | 10 | 10 |
| 22 hr control | 9 | 9 |

In view of the many possible embodiments to which the principles of the disclosure may be applied, it will be recognized that the illustrated embodiments are only preferred examples of the invention and are not to be taken as limiting the scope of the invention.

We claim:

1. A method of generating a recipient cytoplast for nuclear transfer from a pre-metaphase II stage oocyte, comprising
in vitro maturing an oocyte from a non-human mammal,
culturing the oocyte with a microtubule-inhibiting agent, and
enucleating the oocyte.

2. The method of claim 1, wherein the period of in vitro maturation is at least 14 hours.

3. The method of claim 1, wherein the oocyte is derived from a pre-adult non-human mammal.

4. The method of claim 1, wherein the oocyte is derived from an adult non-human mammal.

5. The method of claim 1, wherein the non-human mammal is a cow.

6. The method of claim 1, wherein the oocyte retains a first polar body.

7. The method of claim 1, wherein the microtubule-inhibiting agent is colcemid.

8. The method of claim 7, wherein the concentration of colcemid is at least 0.1 μg/ml.

9. The method of claim 7, wherein the time of colcemid treatment is at least two hours.

10. The method of claim 1, further comprising the steps of:
transferring a single donor cell to the perivitelline space of the recipient cytoplast,
fusing the donor cell with the recipient cytoplast to create a fused oocyte,
activating the fused oocyte,
in vitro culturing a resulting nuclear transfer embryo, and,
transferring the nuclear transfer embryo to a host animal of the same species, wherein a cloned offspring is produced.

11. The method of claim 10, wherein the donor cell is a somatic cell.

12. The method of claim 11, wherein the somatic cell is a fibroblast cell.

13. The method of claim 10, wherein the donor cell is a fetal cell.

14. The method of claim 10, wherein the donor cell is an embryonic cell.

15. The method of claim 10, wherein the in vitro culture of the nuclear transfer embryo is carried out under high oxygen tension.

16. The method of claim 15, wherein the high oxygen tension is a 20% oxygen atmosphere.

17. The method of claim 10, wherein the in vitro culture of the nuclear transfer embryo is carried out in a chemically defined medium.

18. The method of claim 17, wherein the chemically defined medium is CR1aa medium containing fatty acid-free bovine serum albumin.

19. The method of claim 10, wherein the nuclear transfer embryo is transferred to the host animal at the morula stage.

20. The method of claim 10, wherein the nuclear transfer embryo is transferred to the host animal at the blastocyst stage.

21. A recipient cytoplast for nuclear transfer comprising an enucleated pre-metaphase II stage oocyte from a non-human mammal obtained by the process of claim 1.
